(19) United States
(12) Patent Application Publication
(10) Pub. No.: US 2014/0273134 A1
(43) Pub. Date: Sep. 18, 2014

(54) Barley-Based Biorefinery Process

(71) Applicants: DANISCO US INC., Palo Alto, CA (US); The United States of America as represented by the Secretary of Agriculture, Washington, DC (US)

(72) Inventors: Nhuan P. Nghiem, Lansdale, PA (US); Kevin B. Hicks, Malvern, PA (US); David B. Johnston, Wyndmoor, PA (US); Justin M. Montanti, Philadelphia, PA (US); Jayarama K. Shetty, Pleasanton, CA (US)

(73) Assignees: DANISCO US INC., Palo Alto, CA (US); The United States of America as represented by the Secretary of Agriculture, Washington, DC (US)

(21) Appl. No.: 14/204,789

(22) Filed: Mar. 11, 2014

Related U.S. Application Data
(60) Provisional application No. 61/785,997, filed on Mar. 14, 2013.

(51) Int. Cl. C12P 7/14

(52) U.S. Cl. CPC C12P 7/14
USPC 435/162

(57) ABSTRACT

The barley-based biorefinery process comprises a method of optimizing the production of ethanol and value-added products from barley feedstock. Specifically, the biorefinery process is an integrated barley treatment process that utilizes essentially all components of barley (including the barley hulls) to efficiently produce ethanol and other value-added liquids and solids.

### REFERENCE TO RELATED APPLICATIONS

This application claims the benefit of U.S. Provisional Application No. 61/785,997 filed Mar. 14, 2013, which is incorporated herein by reference in its entirety.

### FIELD OF THE INVENTION

The present invention relates to an integrated process for producing bio-fuel and useful chemicals from barley. Specifically, the invention relates to a method for processing barley husks so that an optimal amount of fermentable sugars is extracted for the production of ethanol and other value-added products.

### BACKGROUND OF THE INVENTION

In addition to its other uses, barley is a potential feedstock for bio-fuel production. The use of barley for ethanol production offers several advantages over other bio-fuel crops. Barley can be grown in areas that are not suitable for more commonly grown commercial crops. Winter barley can be double-cropped with corn and soybeans to give farmers three crops in each two-year cycle, thereby further increasing farm productivity. Winter barley is also an important cover crop. Winter barley prevents loss of nitrates, phosphates, and sediment into watersheds and thereby protects the environment and enhances the soil for future crops. Increasing the use of barley can benefit farmers (and the rural economy) outside the “corn belt” by allowing farmers to maximize the potential productivity of their available land.

However, the large-scale use of barley for ethanol production presents a number of challenges. For example, commonly available commercial barley is a relatively low-starch crop. To address this issue, researchers have developed barley species with higher starch contents. Barley grains also contain beta-glucan, which can be hydrolyzed with commercial enzymes called beta-glucanases to produce glucose, which in turn can be used for ethanol production, in addition to the glucose that comes from the starch component of the grains. Even in the grains of the improved barley species, the total starch plus beta-glucans is still lower than the typical starch content in corn, thus resulting in lower final ethanol concentrations in a typical fermentor.

In conventional barley fermentation processes, the barley hulls take up potentially productive space in the fermentor and negatively affect the efficiency of the fermentation process. To increase starch loading, the hulls can be removed and the de-hulled barley grains then used for preparation of the mash used for fermentation. The removed barley hulls are generally discarded as a waste by-product or are simply burned as a fuel to generate heat energy.

However, the removed barley hull fraction still contains some useable starch. The cellulose and hemicellulose components of the barley hulls can be processed and hydrolyzed with commercial enzymes to produce fermentable sugars, which consist of mostly glucose, xylose, galactose, and arabinose. These fermentable sugars can be used as substrates in fermentation processes for production of valuable products, including ethanol and industrial chemicals. Barley hulls also contain the enzyme beta-amylase, which hydrolyzes starch to maltose. This two-glucose molecule can be readily fermented by the commercial yeast Saccharomyces cerevisiae to produce ethanol. Thus, the endogenous beta-amylase in barley hulls can be used to hydrolyze residual starch in the hulls to fermentable maltose and also can be used in a mashing operation where it can help reduce the required dosage of other starch hydrolytic enzymes, in particular glucoamylase.

Presently, only the starch fraction of the barley kernel is considered useful for the production of biofuels and/or useful chemicals. Thus, a need exists for an integrated process that utilizes all fractions, including fiber, of the barley kernel to produce ethanol as well as high value products. The need for such a process exists to convert all fractions of the barley kernel into revenue-generating streams, as previously the non-starch fractions have been treated as waste products. To meet this need, a novel process is developed whereby barley hulls are converted into glucose, which can be used in (among other things) the bio-fuel production process, to produce additional ethanol and other fermentable sugars as well as other value-added co-products.

The need also exists for a process whereby the endogenous beta-amylase of barley hulls is used to reduce the required dosages of other starch hydrolytic enzymes, thus reducing operating costs of barley ethanol fermentation. The current invention comprises an integrated barley biorefinery process whereby significant amounts of glucose and other fermentable sugars are produced from the barley hulls. The glucose may be converted into ethanol or used to produce other value-added products. The value-added products can also be produced from the other fermentable sugars in the invented process. In the invented process the endogenous beta-amylase also is used for partial replacement of some starch hydrolytic enzymes.

The need also exists to reduce costs associated with the purchase of feedstock. This need is met via the disclosed process by the option to utilize the fermentable sugars liberated from the hulls and other fractions to produce additional ethanol while simultaneously lowering the quantity of barley kernels utilized, such that the total ethanol output of the facility is unchanged but the feedstock consumption is reduced.

The need exists to process the hulls in the disclosed manner after separation from the kernel due to the fact that the harsh conditions encountered in the disclosed methods lead to the destruction of starch. Thus, treating the hulls separately from the kernel offers the advantage of minimizing starch loss by converting the maximum possible amount of starch to valuable fuels or chemicals, whereas treating the whole kernel prior to separation of hulls would lead to an unacceptably high reduction in yield.

### SUMMARY OF THE INVENTION

The current invention comprises a method of processing barley to produce ethanol and value-added products. In accordance with the method described herein, the barley hulls are first separated from the endosperm by a conventional dehulling method. The starch is removed from the hulls either by treatment with alpha amylase and glucoamylase to produce a glucose solution, or with endogenous beta-amylase to produce a maltose solution, and destarched hulls. The destarched hulls are pretreated by soaking the hulls in aqueous ammonia, or soaking in ethanol and aqueous ammonia, or by treatment of the hulls having low moisture contents with anhydrous ammonia. The hulls are then hydrolyzed with hemicellulases to produce a xylose solution and residual solids.

A solid/liquid separation process is initiated (e.g. by centrifugation or filtration) to separate the hydrolysate (i.e. the xylose solution) and the residual solids. The residual solids are further hydrolyzed with cellulases to produce a glucose solution. The glucose solution is either used as process water or mixed with glucose or maltose solutions obtained earlier in the refining process and the mixture subsequently is used as process water to prepare a mash of the dehulled barley (endosperm). The mash, containing fermentable glucose or/and maltose from up to and including all three sources (starch from hulls, residual cellulose solids, and starch in endosperm), is used in a fermentation process that utilizes the yeast Saccharomyces cerevisiae to produce ethanol.

The ethanol produced may increase the facility's ethanol output or may allow a reduced feedstock consumption to maintain the same output. The xylose solution produced by the process is used for production of value-added products such as xylitol, astaxanthin, D-ribose, citric acid, lactic acid, butyric acid, itaconic acid, and many others. The xylose may also be converted to xylulose by a commercial enzyme. The xylulose solution may then be fermented to ethanol by Saccharomyces cerevisiae in the same manner as the glucose and/or maltose solutions above.

### BRIEF DESCRIPTION OF THE DRAWINGS

FIG. 1 is a schematic flow chart of the process of the current invention.

FIG. 2 shows pH and A₄₆₅ of wash waters in washing and recovery of destarched barley hulls after ammonia pretreatment as described in Example 9.

FIG. 3 shows ethanol production from mash containing 23 wt % solids of dehulled barley—Comparison of pretreated destarched barley hulls cellulase and hemicellulase hydrolysate vs. de-ionized water (control) used for mashing as described in Example 9.

FIG. 4 shows pH and A₄₆₅ of wash waters in washing and recovery of destarched barley hulls after ammonia pretreatment as described in Example 10.

FIG. 5 shows ethanol production from mash containing 23 wt % solids of dehulled barley—Comparison of pretreated destarched barley hulls cellulase and hemicellulase hydrolysate vs. deionized water (control) use for mashing as described in Example 10.

FIG. 6 shows astaxanthin production using thin stillage obtained from ethanol fermentation broths using dehulled barley mashed in pretreated destarched barley hulls cellulase and hemicellulase hydrolysate vs. de-ionized water (control) as described in Example 10.

FIG. 7 shows the results of ethanol experiments discussed in Example 11 comparing the fermentation of mashes prepared with a combined solution of pretreated destarched barley hull cellulase hydrolysate and wash water vs. de-ionized water (control).

FIG. 8 shows the results of the hydrolysis of liquefied starch, ie “Liquefact”, by endogenous beta-amylase in ground barley hulls.

FIG. 9 shows the results of simultaneous saccharifiaction and fermentation of “Liquefact” using endogenous beta-amylase in barley hulls as enzyme source for maltose production.

FIG. 10 shows weight loss in simultaneous saccharification and fermentation flasks using barley hulls as a source of beta-amylase to replace some of the glucoamylase (FERMENZYME® L-400, DuPont Industrial Biosciences) requirement for hydrolysis of starch in dehulled barley.

FIG. 11 shows final ethanol concentrations obtained in the flasks used to obtain the results shown in FIG. 10.

### DETAILED DESCRIPTION OF PREFERRED EMBODIMENTS

The present invention comprises a method of processing barley to co-produce ethanol and value-added products. In the preferred embodiment, the barley hulls are pretreated and hydrolyzed to generate separate solutions of fermentable sugars, which include a glucose-rich solution and a xylose-rich solution. The glucose in the glucose-rich solution plus the starch in the dehulled barley kernels are used to produce fuel ethanol. The sugars in the xylo se-rich solution are used to produce value-added co-products.

As generally shown in FIG. 1, after barley is harvested it is dehulled. Barley kernels have multiple different uses, however, in the preferred embodiment of the current invention, the kernels are processed into ethanol.

After the removal of the barley kernels (i.e. the endosperm), the barley hulls are treated with alpha amylase and glucoamylase to extract starch (in the form of glucose) from the hulls.

In the preferred embodiment, to take advantage of the endogenous beta-amylase the hulls can be simply soaked in water to cause release of the enzyme, which hydrolyzes some of the starch associated with the hulls to maltose, which is fermentable by the yeast Saccharomyces cerevisiae to produce ethanol, as described in Examples 12-16. In an alternative embodiment, the hulls are mixed with water or a buffer solution (at suitable pH level) and the aforementioned enzymes are added. The mixture is maintained at suitable temperatures and the enzymatic hydrolysis is allowed to proceed until most if not all of the starch in the barley hulls is converted to glucose. The liquid and the residual solids are separated by a common solid/liquid separation technique such as centrifugation or filtration. The liquid, which contains glucose, is saved for further use.

Alternatively, untreated hulls can be added directly to the barley mash as a source of beta-amylase, which will help to reduce the required dosage of the enzyme glucoamylase needed during mashing.

The residual solids are washed with water to extract more glucose and the solid/liquid separation operation is performed. Washing of the residual solids followed by solid/liquid separation are repeated until at least about half of the expected glucose is recovered in the liquids. The solutions that contain the extracted glucose are directed to a conventional ethanol production area.

In another embodiment, the starch in the hulls is liquefied using a suitable method and then incubated in the presence of the hulls at a pH and temperature under which the endogenous beta-amylase present in the hulls will convert the starch to a maltose solution, which may be directed to a conventional ethanol production area. Optionally, a pullulanase or other suitable debranching enzyme may be added to the liquefied starch and hull mixture to increase the concentration of maltose in the resulting solution.

The destarched barley hulls are then pretreated by soaking in aqueous ammonia (SAA) or soaking in ethanol and aqueous ammonia (SEAA) or low moisture anhydrous ammonia process (LMAA). The pretreatment process facilitates enzyme hydrolysis. The pretreated hulls are then hydrolyzed with enzymes containing high levels of hemicellulase such as ACCELLERASE® XY (DuPont Industrial Biosciences) to produce a xylose-rich solution. Although conventional fermentation yeast (e.g. Saccharomyces cerevisiae) cannot metabolize xylo se, many other microorganisms can. These xylose-metabolizing organisms utilize xylose as the main carbon source for growth and production of many industrially important products. Examples of these products include lactic acid, succinic acid, citric acid, itaconic acid, xylitol, astaxanthin, D-ribose, and many others.

In the preferred embodiment, the xylose-rich solution obtained by hydrolysis of the pretreated barley hulls with enzymes containing high levels of hemicellulase are used for production of one or more of these products by using suitable microorganisms that can metabolize the sugars in the xylose-rich solution to produce the desired products. In another embodiment, the xylose may be converted to xylulose by a commercial enzyme. The xylulose may then be fermented to ethanol by Saccharomyces cerevisiae.

The residual solids remaining after hydrolysis with hemicellulase containing enzymes are enriched in cellulose, and are further hydrolyzed with enzymes containing high levels of cellulase, such as ACCELLERASE® 1000 (DuPont Industrial Biosciences), ACCELLERASE® 1500 (DuPont Industrial Biosciences), and ACCELLERASE® XC (DuPont Industrial Biosciences), to produce a glucose-rich solution. Again, solid/liquid separation such as filtration or centrifugation is performed to separate this glucose-rich solution and the final residual solids, which contain mostly lignin and little residual carbohydrates. This glucose-rich solution together with the glucose or maltose solution obtained in the destarching of the barley hulls and the wash waters that are used to extract more glucose from the destarched barley hulls are used as process water to prepare the mash of the dehulled barley for use in the fermentation process for ethanol production using the yeast Saccharomyces cerevisiae.

The use of these glucose or maltose-rich solutions instead of plain process water, which contains no glucose or maltose, result in higher production of ethanol in addition to the ethanol produced solely from the starch in the dehulled barley. Optionally, the amount of dehulled barley utilized in the mash may be reduced such that the total amount of ethanol produced from the dehulled barley and glucose or maltose solutions is the same as would be produced from a greater amount of dehulled barley alone, as described in Example 5, thereby reducing feedstock cost. During the mashing process some untreated barley hulls can be added to the mash as a source of the enzyme beta-amylase, which will help to reduce the required dosage of glucoamylase.

### EXAMPLES

The examples described infra further illustrate the processes of the current invention.

### Example 1

Barley hulls (BH) were dried in an oven at 65° C. overnight. The dried BH contained 17.24% starch on dry basis. 20 g dried BH was placed in a glass bottle and 200 g of 15 wt % NH₄OH was added. The bottle was tightly capped and placed in an incubator at 65° C. Several bottles were prepared as described. The bottles were kept in the incubator for 6, 8, and 24 hours before they were removed and placed in a fume hood. The bottles were allowed to cool for about 15 minutes before the caps were removed. The treated BH was recovered by vacuum filtration using a Whatman filter paper #4. The recovered solids were washed with de-ionized (DI) water until ammonia odor was no longer detected. The washed solids were dried and weighed before their starch contents were determined by standard enzymatic procedure. The residual starch contents, which are expressed as % of starch content in the original (untreated) BH, are as shown in Table 1 (below):

TABLE 1

| | Residual starch |
|---|---|
| Sample | (%, dry basis) |
| Untreated | 100 |
| 6-h ammonia treatment | 58.3 |
| 8-h ammonia treatment | 52.6 |
| 24-h ammonia treatment | 52.6 |

The results show that about one half of the initial starch content in BH was lost in aqueous ammonia treatment. Therefore, recovery of starch should be done before ammonia pretreatment.

### Example 2

Approximately 400 g dry BH (433.88 g at 7.81% moisture) were placed in a beaker. DI water was added to 2000 g total weight. The pH was adjusted to 5.0 with 5 N H₂SO₄. 36.4 ul SPEZYME® Xtra (Thermostable alpha-amylase, DuPont Industrial Biosciences) was added (0.1 kg enzyme/ton dry solids). The slurry was heated to 95° C. and maintained at that temperature with mixing for two hours. Water loss by evaporation was compensated for by addition of DI water to the beaker. The beaker was cooled to 55° C. and 72.7 ul FERMENZYME® L400 (protease and glucoamylase mixture, DuPont Industrial Biosciences) was added (0.2 kg enzyme/ton dry solids). The beaker was maintained at 55° C. overnight. The slurry then was centrifuged at 12,000 rpm for 30 min. 224.4 g destarch water (DSW) was collected. The glucose concentration of the DSW was determined by HPLC. The solid cake was washed with 224.4 g DI water. The slurry was stirred thoroughly and then centrifuged using the same conditions as described previously. The supernatant (SN) was recovered and its glucose concentration determined. The washing step was repeated three times. The results are summarized below in Table 2.

TABLE 2

| | | Glucose | Glucose |
|---|---|---|---|
| | Supernatant | concentration | recovered |
| Sample | (ml) | (g/l) | (g) |
| Destarch water (DSW) | 224.1 | 46.8 | 10.5 |
| Washwater (1st Wash) | 212.0 | 39.6 | 8.4 |
| Washwater (2nd Wash) | 222.4 | 34.6 | 7.7 |
| Washwater (3rd Wash) | 220.0 | 29.6 | 6.5 |

Total glucose recovered (in the DSW plus the three wash waters) was 33.1 g or 43.2% yield (76.5 g glucose is expected to be produced from complete hydrolysis of the starch content of 400 g dry BH). The final residual solid was dried in a 55° C. oven.

### Example 3

Approximately 400 g dry BH was destarched as described in Example 2. The DSW recovered was 228.0 ml and contained 41.8 g/l glucose. The destarched BH (DSBH) was washed with different amounts of water. In Example 2, water was used at 1.64 g/g dry original BH in each wash. The volumes of water used for solid washing in this example were 1.64, 2, 3, 4, and 10 g/g dry original BH. Each experiment was performed using 10 g wet DSBH and in duplicate. After each wash the solid and liquid were separated by centrifugation as described previously. Glucose concentrations in the wash waters were determined by HPLC. The results are summarized in Table 3 below.

TABLE 3

| | | Glucose | | |
|---|---|---|---|---|
| | Super- | concen- | Glucose | Total glucose |
| | natant | tration | recovered | recovered |
| Sample | (ml) | (g/l) | (g) | (g) |
| Destarch water | 228.0 | 41.8 | 9.53 | |
| (DSW) | | | | |
| 1.64 g water/g BH | 2.7 | 28.7 | 13.15 | 22.68 (29.7%) |
| 2 g water/g BH | 3.75 | 26.8 | 17.06 | 26.6 (34.8%) |
| 3 g water/g BH | 5.4 | 22.6 | 20.72 | 30.3 (39.5%) |
| 4 g water/g BH | 6.95 | 19.5 | 23.01 | 32.54 (42.5%) |
| 10 g water/g BH | 21.0 | 10.7 | 38.15 | 47.7 (62.3%) |

The numbers in the parentheses in the last column of the table above are the sum starch in the original BH.

### Example 4

Approximately 400 g dry BH was destarched as described in Example 2. The DSW contained 35.7 g/l glucose. This DSW was used to prepare a mash of dehulled barley (DHB) as follows. 180 g dry DHB was placed in a beaker. The DSW was added to 600 g total weight, i.e. 30% total solids on dry basis. The pH of the slurry was adjusted to 5.2 with 5N H₂SO₄. Then 21.3 ul OPTIMASH® BG (beta-glucanase, DuPont Industrial Biosciences) and 49.1 ul SPEZYME® Xtra (Thermostable alpha-amylase, DuPont Industrial Biosciences) were added. The slurry was maintained at 90° C. for two hours. Mixing was provided by a mechanical agitator. Loss of water due to evaporation was compensated for by the addition of DI water. The slurry then was cooled to 32° C. and its pH adjusted to 3.8 with 5N H₂SO₄. Then 106 ul FERMENZYME® L-400 (glucoamylase plus protease blend, DuPont Industrial Biosciences) and 99.8 ul beta-glucosidase were added together with 0.24 g urea (to give final urea concentration of 0.4 g/kg total mash). The enzyme dosages in terms of kg/ton dry solids are shown below in Table 4:

TABLE 4

| | Dosage |
|---|---|
| Enzyme | (kg/ton total dry solids) |
| OPTIMASH ® BG | 0.13 |
| SPEZYME ® Xtra | 0.30 |
| FERMENZYME ® L-400 | 0.65 |
| Beta-Glucosidase | 0.61 |

The slurry then was transferred into three 250-ml flasks at 150 g/flask. Each flask was inoculated with 0.75 ml of 5% w/v Ethanol Red dry yeast that had been rehydrated in DI water for 30 minutes. The flasks were incubated in a 32° C. incubator and shaken at 190 rpm. A second set of experiments in which the DSW was replaced by DI water was performed following the same procedure. Samples were taken for analysis by HPLC. The ethanol results (% v/v) are summarized below in Table 5.

TABLE 5

| | Ethanol after | Ethanol after |
|---|---|---|
| Mashing liquid | 72-h (% v/v) | 96-h (% v/v) |
| DI water | 17.60 | 17.8 |
| Destarch water (DSW) | 18.50 | 19.6 |

The results demonstrated that additional ethanol could be produced from the glucose in the DSW, which was generated by hydrolysis of the starch in the BH.

### Example 5

Experiments were performed using the same procedure described in Example 4. The enzyme dosages (in kg/ton), urea concentration, and yeast inoculum volume were the same as described above. In the experiments that the DSW was used for mashing the total dry solids was 23% whereas in those that DI water was used for mashing the total dry solids was 27%. The ethanol results are summarized below in Table 6:

TABLE 6

| | Ethanol after | Ethanol after | Ethanol after |
|---|---|---|---|
| Mashing liquid | 48-h (% v/v) | 72-h (% v/v) | 96-h (% v/v) |
| DI water (27% dry solids) | 13.8 | 14.90 | 15.1 |
| Destarch water (DSW) | 13.1 | 15.10 | 15.5 |
| (23% dry solids) | | | |

The results demonstrated that less DHB could be used to produce the same quantities of ethanol using the DSW, which contained the glucose obtained from hydrolysis of starch in the BH, for mashing.

### Example 6

Approximately 70 g dry destarched barley hull (DSBH) was mixed with 700 g 15 wt % NH₄OH (solid:liquid ratio of 1:10) in a 1-liter glass bottle. The bottle was tightly capped then put in an incubator at 65° C. for 8 hours. Two experiments were performed in exactly the same manner. At the end of the experiment the bottles were removed from the incubator and allowed to cool for 1 hour. The solid and liquid then were separated by centrifugation. The liquid was discarded and the solid was washed with DI water of volume equal to the volume of the discarded liquid. The mixture again was centrifuged and the supernatant discarded. The washing step was repeated five times. The compositions of the untreated DSBH and ammonia pretreated destarched barley hull (PDSBH) were determined by the standard procedure developed the National Renewable Energy Laboratory (NREL/LAP-510-42618) and are summarized below in Table 7, which also revealed higher content of all three sugars due to removal of non-carbohydrate components, such as lignin.

TABLE 7

| | Component (wt %, dry basis) | | |
|---|---|---|---|
| Material | Glucan | Xylan | Arabinan |
| Destarched barley hulls (DSBH) | 33.12 | 22.87 | 5.63 |
| SAA-treated DSBH batch 1 | 35.67 | 28.16 | 6.54 |
| SAA-treated DSBH batch 2 | 37.41 | 27.61 | 6.32 |

### Example 7

Batch 1 of the PDSBH described in Example 6 was used in the experiments described in this example. In each experiment appropriate amounts of solid were placed in 50 mM citric acid buffer at pH 5 to give a concentration of 3% (w/v) dry solid.

Enzymes were added as described below:

MULTIFECT® Xylanase (MX) at 1 ml/g xylan

MX+OPTIMASH® BG (regular beta-glucanase)
10 Units/g xylan
50 Units/g xylan

MX+OPTIMASH® TBG (thermo stable beta-glucanase)
10 Units/g xylan
50 Units/g xylan

OPTIMASH® BG
10 Units/g xylan
25 Units/g xylan
50 Units/g xylan

No Enzyme (control)

Each experiment was performed with 10 g slurry (solid plus buffer) in 50-ml plastic tubes which were tightly capped and incubated with shaking in an incubator at 50° C. for 72 hours. The experiments were performed in duplicate and the average results are described below in Table 8:

TABLE 8

| Enzyme treatment | Xylose yield (% of theoretical yield) |
|---|---|
| MX | 38.0 |
| MX + OPTIMASH ® TBG | 38.1 |
| at 10 units/g xylan | |
| MX + OPTIMASH ® TBG | 42.8 |
| at 50 units/g xylan | |
| MX + OPTIMASH ® BG | 32.7 |
| at 10 units/g xylan | |
| MX + OPTIMASH ® BG | 34.4 |
| at 50 units/g xylan | |
| OPTIMASH ® BG | 2.6 |
| at 10 units/g xylan | |
| OPTIMASH ® BG | 4.3 |
| at 25 units/g xylan | |
| OPTIMASH ® BG | 5.5 |
| at 50 units/g xylan | |
| No enzyme (control) | 0.5 |

The results demonstrated that after pretreatment with ammonia xylose could be obtained from the PDSBH by hydrolysis using commercial xylanase alone or xylanase plus beta-glucanase.

### Example 8

Batch 1 of the PDSBH described in Example 6 was used in the experiments described in this example. In each experiment appropriate amounts of solid were placed in 50 mM citric acid buffer at pH 5 to give a concentration of 3% (w/v) dry solid. The enzymes used were ACCELLERASE® 1000 (cellulase, DuPont Industrial Biosciences), ACCELLERASE® 1500 (cellulase, DuPont Industrial Biosciences), ACCELLERASE® XC (cellulase, DuPont Industrial Biosciences), ACCELLERASE® XY (xylanase, DuPont Industrial Biosciences) and MULTIFECT® Xylanase (xylanase, DuPont Industrial Biosciences). Each enzyme was used at three dosages, which were 0.05, 0.1, and 0.25 ml/g dry biomass. Each experiment was performed with 10 g slurry (solid plus buffer) in 50-ml plastic tubes which were tightly capped and incubated with shaking in an incubator at 50° C. for 72 hours.

The experiments were performed in duplicate and the average results are described below in Table 9:

TABLE 9

| | Enzyme | | |
|---|---|---|---|
| | dosage | Glucose yield | |
| | (ml/g dry | (% of theoretical | Xylose yield (% of |
| Enzyme treatment | biomass) | yield) | theoretical yield) |
| ACCELLERASE ® | 0.05 | 25.5 | 14.45 |
| 1000 | 0.1 | 34.75 | 19.07 |
| | 0.25 | 46.45 | 25.48 |
| ACCELLERASE ® | 0.05 | 19.39 | 9.92 |
| 1500 | 0.1 | 27.32 | 13.62 |
| | 0.25 | 40.56 | 20.29 |
| ACCELLERASE ® | 0.05 | 23.6 | 14.05 |
| XC | 0.1 | 30.69 | 22.12 |
| | 0.25 | 43.55 | 39.09 |
| ACCELLERASE ® | 0.05 | 4.97 | 34.74 |
| XY | 0.1 | 6.4 | 39.81 |
| | 0.25 | 9.77 | 45.27 |
| MULTIFECT ® | 0.05 | 4.32 | 21.39 |
| Xylanase | 0.1 | 5.95 | 30.72 |
| | 0.25 | 9.13 | 41.65 |

The results demonstrated that after pretreatment with ammonia the PDSBH could be hydrolyzed with commercial cellulase products, which also contain some xylanase activity, to make glucose-rich solutions, or with commercial xylanase products to make xylose-rich solutions.

### Example 9

Destarched barley hull (DSBH) was pretreated with 15 wt % NH₄OH as described in Example 6, except the pretreatment time was 16 hours instead of 8 hours. After the pretreatment the solids were recovered and washed as described in Example 6. The pH and absorbance at 465 nm, which is the wavelength normally used for color determination of wastewaters in Kraft paper mills, were measured for each wash water. The pH and A₄₆₅ results are shown in FIG. 2.

The results showed the decrease of pH and color, which was an indication of lignin solubilized during the ammonia pretreatment, after each wash. The compositions of the DSBH and PDSBH are summarized below in Table 10, which also revealed higher content of all three sugars due to removal of non-carbohydrate components, such as lignin.

TABLE 10

| | Component | | |
|---|---|---|---|
| | (wt %, dry basis) | | |
| Material | Glucan | Xylan | Arabinan |
| Destarched barley hulls | 37.23% | 23.78% | 5.13% |
| (DSBH) | | | |
| SAA-treated DSBH | 46.72% | 29.56% | 6.47% |

Approximately 40 g (dry solids basis) of the solids recovered after the last wash was placed in a flask to which 50 mM citric acid buffer at pH 5 was added to make a slurry of 5% w/v dry solids. The pH was readjusted to 5 with 2N sulfuric acid. Two enzyme products ACCELLERASE® 1000 (DuPont Industrial Biosciences) and ACCELLERASE® XY (DuPont Industrial Biosciences) were added each at a dosage of 0.25 ml/g dry biomass. The flask then was placed in an incubator at 50° C. with shaking at 200 rpm for 72 hours. The liquid was recovered by centrifugation. The sugar concentrations in the recovered liquid were 18.3 g/l glucose and 9.1 g/l xylose, which were equivalent to 65% and 50% theoretical yields, respectively. This sugar solution was used to prepare a mash of dehulled barley (DBH) as described in Example 4. The total solids of the mash was 23 wt %. A control set of experiments which used DI water for mashing was also performed in parallel. Each set of experiments was performed in triplicate. The averaged ethanol results (% v/v) are summarized below in Table 11.

TABLE 11

| | Ethanol concentration | | | | |
|---|---|---|---|---|---|
| | (% v/v) | | | | |
| Mashing Liquid | 0 h | 24 h | 47 h | 72 h | 136 h |
| Cellulase and Hemicellulase | 0.0 | 0.3 | 10.0 | 12.6 | 13.3 |
| Hydrolysate | | | | | |
| Water (Control) | 0.0 | 4.9 | 10.2 | 11.7 | 12.0 |

The average ethanol results are plotted in FIG. 3.

The results demonstrated that at the same dehulled barley solid loadings more ethanol was obtained when the hydrolysate was used for mashing due to the glucose present in the hydrolysate, which served as an extra substrate for the fermentation. The results also indicated that although in the experiments using the hydrolysate for mashing the yeast suffered a short lag it eventually caught up with and surpassed the control experiments where DI water was used for mashing. The lag period could be caused by inhibitory compounds formed during the ammonia pretreatment. However, the results showed that after acclimation the yeast was able to overcome the initial inhibition and fermented glucose to ethanol at high efficiency.

### Example 10

Destarched barley hull (DSBH) was pretreated with 15 wt % NH₄OH as described in Example 9. After the pretreatment the solids were recovered and washed as described in Example 9. The pH and absorbance at 465 nm, which is the wavelength normally used for color determination of wastewaters in Kraft paper mills, were measured for each wash water. The pH and A₄₆₅ results are shown in FIG. 4.

The results showed the decrease of pH and color, which was an indication of lignin solubilized during the ammonia pretreatment, after each wash. The compositions of the DSBH and PDSBH are summarized below in Table 12 which also revealed higher content of all three sugars due to removal of non-carbohydrate components, such as lignin.

TABLE 12

| | Component | | |
|---|---|---|---|
| | (wt %, dry basis) | | |
| Material | Glucan | Xylan | Arabinan |
| Destarched Barley Hull (DSBH) | 37.23% | 23.78% | 5.13% |
| SAA-tretreated DSBH Batch 1 | 48.25% | 29.41% | 6.05% |
| SAA-tretreated DSBH Batch 2 | 49.32% | 28.99% | 5.99% |

The entire batch 1 of the PDSBH was hydrolyzed with enzymes in 50 mM citric acid buffer at pH 5 and 50° C. as described in Example 9. The solid concentration was 7.75 wt % and the enzymes used were ACCELLERASE® 1000 (DuPont Industrial Biosciences) (0.25 ml/g biomass) and ACCELLERASE® XY (DuPont Industrial Biosciences) (0.25 ml/g biomass). Both enzymes were added together and the hydrolysis was performed for 72 hours. The glucose and xylose concentrations in the hydrolysate were 31.9 and 16.0 g/l, respectively.

This sugar solution was used to prepare a mash of dehulled barley (DBH) as described in Example 4. The total solids of the mash was 23 wt %. A control set of experiments which used DI water for mashing was also performed in parallel. Each set of experiments was performed in triplicate. The averaged results are summarized below in Table 13.

TABLE 13

| | Ethanol concentration (% v/v) | | | | |
|---|---|---|---|---|---|
| Mashing Liquid | 0 h | 24 h | 47 h | 72 h | 136 h |
| Cellulase and Hemicellulase | 0.0 | 7.2 | 11.2 | 14.0 | 14.7 |
| Hydrolysate | | | | | |
| Water (Control) | 0.0 | 7.6 | 11.3 | 11.7 | 12.1 |

The average ethanol results are plotted in FIG. 5.

The results demonstrated that at the same dehulled barley solid loadings more ethanol was obtained when the hydrolysate was used for mashing due to the glucose present in the hydrolysate, which served as an extra substrate for the fermentation. The results also indicated no lag period in the experiments where the hydrolysate was used for mashing.

At the end of the fermentations, the hydrolysate flasks were combined together and the water flasks were combined together. The two combined broths were heated over a hot plate with gentle heating to remove ethanol. After 2 hours about one half of the water in the broths was lost due to evaporation. The ethanol concentrations in the combined hydrolysate broth and the combined water broth were 0.3 and 0.2% v/v, respectively. The broths, which now contained very low levels of ethanol, were used for astaxanthin production. The experiments on astaxanthin production are described next.

To prepare inoculum for astaxanthin production, YM media was prepared using 21 g/l YM powder per instructions by the manufacturer. The media was transferred into two 250-ml flasks (25 ml per flask), which then were autoclaved at 121° C. for 20 minutes. Upon cooling each flask was inoculated with one loopful from a plate of Phaffia rhodozyma JTM 185, which was an astaxanthin-producing organism developed in our own laboratory. The flasks were incubated at 22° C. and 250 rpm. The hydrolysate and water “thin stillage” obtained by boiling off most of the ethanol as described previously were adjusted to pH 5 with 1 N NaOH and transferred into 250-ml flasks (25 ml per flask). Each set of thin stillage experiments were performed in duplicate. The flasks were autoclaved at 121° C. for 20 minutes. The four-day old inoculum was used to inoculate the thin stillage flasks (1 ml inoculum per flask). The thin stillage flasks were incubated at 22° C. and 250 rpm. Samples were taken at 0, 24, 48, 75, and 145 hours.

The averaged dry cell weight results are summarized below in Table 14:

TABLE 14

| | Dry Cell Weight |
|---|---|
| Thin Stillage Source | (g/L) |
| Cellulase and Hemicellulase Hydrolysate Mash | 15.9 |
| Water Mash | 10.4 |

The carotenoid results are plotted in FIG. 6. The xylose concentration in the hydrolysate thin stillage flasks dropped from 18 g/l at the beginning of the experiments to 0.9 gnat 145 hours.

The results indicated that the thin stillage obtained by boiling off ethanol could be used for production of astaxanthin as a value-added co-product of ethanol. The xylose, which was not metabolized by the yeast S. cerevisiae during ethanol production, could be used as a carbon source for cell growth and astaxanthin production.

Astaxanthin is a carotenoid used as a supplement in aquatic feed to give the flesh of farm-raised fish the pink color that the wild fish obtained from eating astaxanthin-containing algae. Astaxanthin also has many health benefits and its market for human consumption may become very large. Astaxanthin was used as an example to demonstrate the feasibility of making a value-added co-product. Other co-products of interest could be produced in the same manner by using suitable xylose-metabolizing organisms. Examples include succinic acid, itaconic acid, butyric acid, lactic acid, citric acid, xylitol, and many others.

### Example 11

Destarched barley hull (DSBH) was pretreated with 15 wt % NH₄OH as described in Example 9. After the pretreatment the solids were recovered and washed as described in Example 9.

Approximately 31 g (dry basis) of the PDSBH was placed in a flask and mixed with appropriate amount of DI water to make a total mass of 310 g (i.e., 10% solids on dry basis). The pH of the slurry was adjusted to 5 with 2N H₂SO₄. ACCELLERASE® XY (xylanase, DuPont Industrial Biosciences) was added at 0.25 ml/g biomass (dry basis). The flask was incubated at 50° C. and 250 rpm for 96 hours then was harvested by centrifugation. 208.1 g hydrolysate and 97.3 g wet solids (moisture 77.80%, thus, 21.6 g dry) were recovered.

The sugar concentrations of the xylanase hydrolysate are summarized below in Table 15. The hydrolysate was rich in xylose and low in glucose.

TABLE 15

| | Glucose | Xylose |
|---|---|---|
| Hydrolysis time (h) | (g/l) | (g/l) |
| 0 | 0.48 | 3.24 |
| 24 | 0.69 | 12.64 |
| 72 | 2.11 | 16.41 |
| 93 | 3.04 | 17.01 |

The solid recovered in the xylanase hydrolysis (the entire 21.6 g dry) was placed in a flask. DI water was added to 216 g total mass. The pH was still at 5 and was not re-adjusted. ACCELLERASE® 1000 (cellulase, DuPont Industrial Biosciences) was added at 0.25 ml/g dry biomass. The flask was incubated at 50° C. and 250 rpm and then was harvested at 76 hours by centrifugation. The sugar concentrations of the cellulase hydrolysate are summarized below in Table 16. This hydrolysate was enriched in glucose and low in xylose.

TABLE 16

| | Glucose | Xylose |
|---|---|---|
| Hydrolysis time (h) | (g/l) | (g/l) |
| 0 | 3.15 | 6.90 |
| 76 | 53.47 | 9.43 |

The residual solids were washed with 216 g DI water (i.e., 10 times the original solid weight). The mixture was incubated at 50° C. and 250 rpm for 1 hour. The wash water was then recovered by centrifugation. This wash water contained 10.44 g/l glucose and 1.81 g/l xylose. The cellulase hydrolysate and wash water were combined. The combined solution contained 28.6 g/l glucose. The combined sugar solution was used to make a mash of 23% dehulled barley for ethanol fermentation as described in Example 10. A control set of experiments also was performed where DI water was used for mashing. Each set of experiments was performed in triplicate. The averaged results of these ethanol fermentation experiments are summarized below in Table 17.

TABLE 17

| | Ethanol concentration (% v/v) | | | | |
|---|---|---|---|---|---|
| Mashing Liquid | 0 h | 24 h | 48 h | 72 h | 137 h |
| Cellulase Hydrolysate and Wash Water | 0.0 | 7.2 | 11.8 | 13.9 | 14.4 |
| Water (Control) | 0.0 | 8.2 | 11.4 | 11.8 | 12.1 |

The results demonstrated that the glucose present in the combined sugar solution (cellulase hydrolysate plus wash water) resulted in additional ethanol production. At the end of the fermentations, the hydrolysate flasks were combined together and the water flasks were combined together. The two combined broths were heated on a hot plate with low heating for 2 hours to remove ethanol. The broths with low ethanol levels (about 0.2% v/v) were centrifuged and the liquids (thin stillage) were collected for astaxanthin production experiments.

To prepare inoculum for astaxanthin production, YM media was prepared using 21 g/l YM powder per instructions by the manufacturer. The media was transferred into two 250-ml flasks (25 ml per flask), which then were autoclaved at 121° C. for 20 minutes. Upon cooling each flask was inoculated with one loopful from a plate of Phaffia rhodozyma JTM 185, which was an astaxanthin-producing organism developed in our own laboratory. The flasks were incubated at 22° C. and 250 rpm. The xylanase hydrolysate (Table 15) was used in the first set of experiments on astaxanthin production. Xylanase hydrolysate was added to three 250-ml flasks at 25 ml per flask. Amberex 695AG yeast extract was also added to the flasks to give final concentration of 5 g/l. The pH was adjusted to 5 and the flasks were autoclaved at 121° C. for 20 minutes. The four-day old inoculum was used to inoculate the xylanase hydrolysate flasks (1 ml inoculum per flask). The flasks were incubated at 22° C. and 250 rpm. Samples were taken for analysis.

The experiment was performed in triplicate and the carotenoid results are summarized below in Table 18, which show astaxanthin production from xylose in the xylanase hydrolysate.

TABLE 18

| | Carotenoid concentration | | | | |
|---|---|---|---|---|---|
| | (mg/l) | | | | |
| Flask | 0 h | 24 h | 48 | 71 | 140 |
| 1 | 0.27 | 0.59 | 2.36 | 5.84 | 8.17 |
| 2 | 0.27 | 0.63 | 2.22 | 5.84 | 8.42 |
| 3 | 0.26 | 0.62 | 2.01 | 5.09 | 8.44 |
| Average | 0.27 | 0.61 | 2.20 | 5.59 | 8.34 |

The xylose concentrations in the samples taken at 0, 24, 48, 71, and 140 hours were 17.2, 17.4, 14.2, 1.7, and 0.2 g/l, respectively. The average final dry cell weight was 4.8 g/l.

In the next set of experiments, the thin stillage obtained from the broths of the ethanol fermentations (see Table 17 above) was combined with equal volumes of the xylanase hydrolysate. The resulting solutions were used for astaxanthin production as described previously. No nutrients were added to these experiments. Each set of experiments was performed in duplicate. The averaged astaxanthin results are summarized below in Table 19. The results show astaxanthin production from the sugars in the combined cellulase hydrolysate obtained from the residue remaining after the hydrolysis by xylanase described previously.

TABLE 19

| | Carotenoid concentration (mg/l) | | | | |
|---|---|---|---|---|---|
| Thin Stillage Source | 0 h | 24 h | 48 h | 72 h | 145 h |
| Cellulase Hydrolysate and | 0.21 | 0.51 | 2.45 | 6.70 | 19.39 |
| Wash Water | | | | | |
| Water (Control) | 0.31 | 0.64 | 2.60 | 7.38 | 15.08 |

### Example 12

Barley hulls were incubated in 50 mM citrate buffer at pH 4.8 at 5 wt % solid loading with the following enzymes: FERMGEN® (protease, DuPont Industrial Biosciences), STARGEN® 002 (native starch hydrolytic enzyme, DuPont Industrial Biosciences), SPEZYME® Xtra (thermostable alpha-amylase, DuPont Industrial Biosciences), and PROTEX® 6L (protease, DuPont Industrial Biosciences). The enzymes were added individually at 1% based total solid. Samples were taken after 24 hours and analyzed for glucose and maltose by HPLC. Since the barley hulls contained 15.18 wt % starch the theoretical yield of starch hydrolysis was 5 wt % solids*15.18 wt % starch*1.11=0.83 wt % glucose plus maltose. The actual yield was calculated as yield=(maltose+glucose)÷0.83.

The results are shown in Table 20.

TABLE 20

| Enzyme | Yield |
|---|---|
| None | 13.05% |
| FERMGEN ® | 13.66% |
| STARGEN ® 002 | 74.55% |
| SPEZYME ® Xtra | 63.44% |
| PROTEX ® 6L | 10.23% |

The results indicated that the endogenous beta-amylase in the barley hulls were sufficient to hydrolyze the native starch in the hulls to achieve about 13% yield of fermentable maltose and glucose. Addition of alpha-amylase (SPEZYME® Xtra, DuPont Industrial Biosciences) and enzyme product capable of hydrolyzing native starch (STARGEN® 002, DuPont Industrial Biosciences) significantly improved starch hydrolysis. On the other hand, addition of proteases (FERMGEN® and PROTEX® 6L, both DuPont Industrial Biosciences) either did not improve or negatively affect starch hydrolysis. The negative effect (PROTEX® 6L, DuPont Industrial Biosciences) probably was due to degradation of some of the endogenous beta-amylase.

### Example 13

Experiments were performed in a similar manner as described in Example 12 except that the citrate buffer was replaced by “liquefact”, which is a solubilized starch solution containing 46 wt % solubilized starch (measured as maltodextrin). The yield was calculated as yield=final (maltose+glucose)÷initial maltodextrin. The results are shown in Table 21.

TABLE 21

| Enzyme | Yield |
|---|---|
| None | 54.41% |
| FERMGEN ® | 48.91% |
| STARGEN ® 002 | 83.63% |
| SPEZYME ® Xtra | 55.79% |
| PROTEX ® 6L | 45.52% |

Since the substrates in this case were solubilized starch, which contained more reducing ends than in the case of native starch (Example 12), the endogenous beta-amylase was more efficient and hydrolyzed starch to 54% of the theoretical value. The addition of alpha-amylase (SPEZYME® Xtra, DuPont Industrial Biosciences) did not improve the hydrolysis whereas STARGEN® 002 (DuPont Industrial Biosciences), which contained glucoamylase, resulted in significant improvement. The addition of proteases (FERMGEN® and PROTEX® 6L, both DuPont Industrial Biosciences) caused small negative effects on the solubilized starch hydrolysis.

### Example 14

Barley hulls (BH) were ground in a coffee grinder. In a 125 ml flask, 0.5 g ground BH were added to 25 ml of enzyme liquefied starch (Liquefact) at pH 5.5. The mixture was incubated at 55° C. with 250 RPM orbital shaking for 24 hours. Starch degradation and maltose production were monitored during the incubation by HPLC. The results are shown in FIG. 8. The results demonstrate that the beta-amylase activity endogenous to the barley hull was sufficient to convert over half of the available starch to maltose in 24 hours, with most of the conversion completed after only 2 hours of incubation.

### Example 15

In the same method described in Example 14, barley hulls (BH) were added to enzyme liquefied starch at pH 5.5 and incubated at 55° C. Incubation was terminated after four hours and the temperature was reduced to 32° C. over the course of 30 minutes. After temperature adjustment, yeast extract was added at a concentration of 5 g/L and the solution was inoculated with 0.125 ml of 5% (w/v) Ethanol Red dry yeast that had been rehydrated in DI water for 30 minutes. The flasks were then incubated at 32° C. with 190 RPM orbital shaking for 46.5 hours. Maltose, maltodextrin, and ethanol concentrations were determined at the start (0 h) and finish (46.5 h) of fermentation. The SSF results are shown in FIG. 9. No significant conversion of maltodextrin to maltose was observed during the SSF, which was expected as the four-hour pre-hydrolysis step was sufficient to achieve the typical maximum level of conversion. The results indicate that most of the available maltose, generated by beta-amylase present in the barley hull, was fermented to produce ethanol at 70% of theoretical yield based on the maltose present at the beginning of the fermentation.

### Example 16

Dehulled barley was used to prepare a mash according to our standard procedure. Ground dehulled barley was added to DI water to make a mash of 1600 g total mass containing 30 wt % solids. The pH was adjusted to 5.2. Two enzymes were added, OPTIMASH® BG (DuPont Industrial Biosciences) at 0.13 g/kg and SPEZYME® Xtra (DuPont Industrial Biosciences) at 0.3 g/kg. The mash was heated to 90° C. and maintained at that temperature for two hours. Then it was cooled and water loss due to evaporation was compensated for by the addition of DI water. The pH was adjusted to 3.8, urea was added at 400 mg/kg and beta-glucosidase was added at 0.61 g/kg. The mash then was mixed thoroughly and divided into four equal portions of 400 g each. Each portion of the mash received different amounts of glucoamylase (FERMENZYME® L-400, DuPont Industrial Biosciences) as follows: none (control experiment), one third of the standard dosage, two thirds of the standard dosage, and the full amount of the standard dosage.

The amounts of FERMENZYME® L-400 (DuPont Industrial Biosciences) added are summarized in Table 22.

TABLE 22

| FERMENZYME ® L-400 | FERMENZYME ® L-400 |
|---|---|
| Dosage (g/kg) | As % of standard dosage |
| 0.65 | 100 |
| 0.429 | 66 |
| 0.215 | 33 |
| 0 | 0 |

After thorough stirring to ensure uniform distribution of enzyme each portion was poured into three 250-ml flasks, each of which received 100 g mash. Ground barley hull was added equally to all flasks at 2 g/flask. Yeast inoculum was prepared by adding 0.5 g Ethanol Red yeast to 9.5 ml DI water and rehydrating for 30 minutes. Each flask was inoculated with 0.5 ml rehydrated yeast. The flasks then were incubated in a shaker maintained at 32° C. Progress of ethanol production was followed by weight loss due to carbon dioxide production. Samples were taken for HPLC analysis of ethanol and other metabolites at the end of the experiments. The average weight loss and final ethanol results are shown in FIG. 10 and FIG. 11, respectively. The results indicate that barley hulls used at 2 wt % of the total dehulled barley mash could replace about one third of the glucoamylase (FERMENZYME® L-400, DuPont Industrial Biosciences) requirement for ethanol production, thus reducing cost.

For the foregoing reasons, it is clear that the invention provides an innovative method of processing barley to co-produce ethanol and value-added products. The invention may be modified in multiple ways and applied in various technological applications. For example, each step may be automated so that automated machinery moves the product progressively through the described process.

The current invention may be modified and customized as required by a specific operation or application, and the individual components may be modified and defined, as required, to achieve the desired result. Although the materials of construction are not described, they may include a variety of compositions consistent with the function of the invention. Such variations are not to be regarded as a departure from the spirit and scope of the invention, and all such modifications as would be obvious to one skilled in the art are intended to be included within the scope of the following claims.

What is claimed is:

1. A method of processing barley to co-produce ethanol and value-added products, the method comprising the steps of:
(a) separating barley hulls from barley endosperm;
(b) treating the hulls with alpha-amylase and glucoamylase to produce a glucose solution and destarched hulls;
(c) treating the destarched hulls to produce pretreated hulls by soaking the destarched hulls in aqueous ammonia, or soaking in ethanol and aqueous ammonia, or by treating with anhydrous ammonia;
(d) hydrolyzing the pretreated hulls with hemicellulases to produce a xylo se solution and residual solids;
(e) further hydrolyzing the residual solids with cellulases to produce a glucose solution;
(f) using the glucose solutions obtained in (b) and (e) as mashing waters to produce ethanol in addition to ethanol produced from starch in the barley endosperm, or optionally to produce a same amount of ethanol from a reduced quantity of barley endosperm; and,
(g) using the xylose solution obtained in (d) for production of value-added products.

2. The method of claim 1 wherein step (b) is replaced by a step comprising treatment of starch within the hulls by endogenous beta-amylase contained in the hulls and optionally other suitable debranching enzymes to produce a maltose solution and destarched hulls.

3. The method of claim 1 wherein between steps (b) and (c) the destarched hulls are washed with water or a buffer to produce additional glucose and an additional solid/liquid separation step occurs before the destarched hulls proceed to step (c).

4. The method of claim 3 wherein the step described in claim 3 is repeated until about half of an expected total amount of glucose is recovered.

5. The method of claim 1 wherein after step (d) and before step (e) the solids are separated from the xylo se by centrifugation or filtration.

6. The method of claim 1 wherein after step (e) an additional solid/liquid separation step is initiated.

7. The method of claim 6 wherein the additional solid/liquid separation step comprises centrifugation or filtration.

8. The method of claim 6 wherein the final residual solids comprise lignin and residual carbohydrates.

9. The method of claim 1 wherein step (f) comprises using the maltose solution obtained in (b) and the glucose solution obtained in (e) as mashing waters to produce additional ethanol in addition to the ethanol produced from the starch in the barley endosperm or optionally to produce a same amount of ethanol from a reduced quantity of barley endosperm.

10. The method of claim 1 wherein, in step (f), yeast is used to ferment the mashing waters.

11. The method of claim 10 wherein the yeast comprises Saccharomyces cerevisiae.

12. The method of claim 1 wherein, in step (f), barley hulls are used as a source of beta-amylase to replace some of the required glucoamylase in the mashing.

13. The method of claim 1 wherein in step (g), the value-added products are selected from a group consisting of astaxanthin, lactic acid, succinic acid, citric acid, itaconic acid, xylitol, ribose, and others.

14. The method of claim 9 wherein, in step (f), Saccharomyces cerevisiae yeast is used to ferment the mashing waters.

15. The method of claim 14 wherein barley hulls are used as a source of beta-amylase to replace some of the required glucoamylase in the mashing.
